(19) United States
(12) Patent Application Publication
(10) Pub. No.: US 2014/0357515 A1
(43) Pub. Date: Dec. 4, 2014

(54) Method and Kit for Identification and Quantification of Single-Strand Target Nucleic Acid

(71) Applicants: Katja Friedrich, Erlenbach a. Main (DE); Walter Gumbrecht, Herzogenaurach (DE); Yiwei Huang, Erlangen (DE); Mathais Sprinzl, Bayreuth (DE)

(72) Inventors: Katja Friedrich, Erlenbach a. Main (DE); Walter Gumbrecht, Herzogenaurach (DE); Yiwei Huang, Erlangen (DE); Mathais Sprinzl, Bayreuth (DE)

(21) Appl. No.: 14/365,789

(22) PCT Filed: Dec. 11, 2012
(86) PCT No.: PCT/EP2012/075093

§ 371 Date: Jun. 16, 2014

(30) Foreign Application Priority Data
Dec. 16, 2011 (DE) 10 2011 088 831.4
Mar. 20, 2012 (DE) 10 2012 204 366.7

(51) Int. Cl. C12Q 1/68

(52) U.S. Cl. CPC C12Q 1/6834; C12Q 1/6825
USPC 506/9; 435/6.11; 506/16

(57) ABSTRACT

A method for identification and quantification of at least one single-stranded target nucleic acid and a kit for detection of at least one single-stranded target nucleic acid in a sample are described. The method includes contacting at least one solid carrier that includes at least one capture oligonucleotide immobilized thereon with at least one complementary-strand oligonucleotide, at least one single-stranded target nucleic acid, and at least one reporter oligonucleotide that includes a label. The target nucleic acid is identified by reading the label of the reporter oligonucleotide on the carrier.

### RELATED APPLICATIONS

This application is the National Stage of International Application No. PCT/EP2012/075093, filed Dec. 11, 2012, which claims the benefit of German Patent Application No. DE 102012204366.7, filed Mar. 20, 2012 and German Patent Application No. DE 102011088831.4 filed Dec. 16, 2011. The entire contents of each of these three documents are hereby incorporated herein by reference.

### TECHNICAL FIELD

The present teachings relate generally to methods for identifying and quantifying at least one single-stranded target nucleic acid and to kits for detecting at least one single-stranded target nucleic acid in a sample.

### BACKGROUND

The expression of genes in cells is tightly controlled and is regulated by diverse molecular processes. In addition to the complex regulation of the transcription of the genes, mechanisms at the posttranscriptional level (e.g., at the mRNA level) are also important. Short, noncoding nucleic acid molecules may intervene in gene expression by attaching in a highly specific manner to complementary sequences of mRNA molecules, thereby regulating translation of the mRNA into the corresponding protein. The short nucleic acid molecules are called microRNAs (“miRNAs” for short). In recent years, miRNAs have been a topic of interest in molecular biology and medical research. For example, it has been demonstrated that some miRNAs are directly associated with the development of certain cancers (e.g., breast cancer, lung cancer, or leukemia). In these cancers, the cancer cells exhibit an increased or reduced number of specific miRNAs. Certain miRNAs also occur in altered concentrations in the case of immunological disorders (e.g., rheumatism). It is thought that over 1000 different miRNA molecules occur in humans alone.

Methods for specifically identifying and quantifying miRNAs are sought due to the growing importance of miRNAs and the multiplicity thereof. Various methods for detecting miRNAs have been used, such as miRNA sequencing or miRNA amplification by real-time PCR.

U.S. Pat. No. 6,322,971 describes a hybridization-based method for detecting a nucleic acid. In this method, the nucleic acid to be detected is covalently bonded to a second, labeled nucleic acid after the two nucleic acids have attached to an immobilized opposite-strand oligonucleotide and missing nucleotides between the nucleic acid to be detected and the labeled nucleic acid have been filled in by a DNA polymerase. Nonbonded labeled nucleic acids are removed by increasing the temperature.

U.S. Pat. No. 6,344,316 describes a method for detecting nucleic acids wherein an immobilized capture oligonucleotide is covalently bonded to a second, labeled oligonucleotide after the oligonucleotides have attached as an opposite strand to the nucleic acid to be detected. After removal of the nucleic acid to be detected by washing at high temperature, the covalently bonded label remains.

Various problems have been observed in conventional methods. For example, the specificity and the sensitivity of miRNA detection may be inadequate. In the case of hybridization-based methods, the low specificity is due inter alia to the fact that full hybridization of the miRNA is not a prerequisite for the identification process. Because of the low sensitivity of the methods, the miRNAs may be amplified before their detection. During amplification, incorporation of incorrect nucleotides may occur, thereby even further limiting the specificity of miRNA detection. In addition, the methods are carried out at low temperatures because the small length of miRNA hybrids is associated with low melting temperatures. As a result, miRNA detection is not especially robust or quantitatively analyzable. Moreover, many methods require pre-analytical miRNA labeling, and may entail the risk of inaccurate results and artifacts.

### SUMMARY AND DESCRIPTION

The scope of the present invention is defined solely by the appended claims, and is not affected to any degree by the statements within this summary.

The present embodiments may obviate one or more of the drawbacks or limitations in the related art. For example, in some embodiments, a method that solves one or more of the problems observed in conventional methods, and a kit for carrying out the method, are provided.

A method in accordance with the present teachings for identifying and quantifying at least one single-stranded target nucleic acid includes: providing at least one solid support that includes at least one capture oligonucleotide immobilized thereon; and contacting the support under a first reaction condition with at least one opposite-strand oligonucleotide, at least one single-stranded target nucleic acid, and at least one reporter oligonucleotide having a label. The opposite-strand oligonucleotide includes an oligonucleotide sequence at least sectionally complementary to the capture oligonucleotide, an oligonucleotide sequence complementary to the target nucleic acid, and an oligonucleotide sequence at least sectionally complementary to the reporter oligonucleotide. The opposite-strand oligonucleotide is configured for hybridizing at least sectionally each of the capture oligonucleotide and the reporter oligonucleotide, and is further configured for hybridizing the target nucleic acid to the opposite-strand oligonucleotide. A first end of the target nucleic acid and a free end of the capture oligonucleotide are configured to form base pairings with adjacent nucleotides of the opposite-strand oligonucleotide. A second end of the target nucleic acid and a first end of the reporter oligonucleotide are configured to form base pairings with adjacent nucleotides of the opposite-strand oligonucleotide. A method in accordance with the present teachings further includes incubating the support under the first reaction condition; ligating the first end of the target nucleic acid to the free end of the capture oligonucleotide to covalently bond the target nucleic acid to the capture oligonucleotide, and ligating the second end of the target nucleic acid to the first end of the reporter oligonucleotide to covalently bond the target nucleic acid to the reporter oligonucleotide; incubating the support under a second reaction condition, such that the reporter oligonucleotide remains connected to the support when the target nucleic acid ligated at the first end and the second end thereof; and reading the label of the reporter oligonucleotide on the support.

The phrase “target nucleic acid” refers to a single-stranded nucleic acid of about 10 to about 30 nucleotides in length. The nucleic acid may be an RNA or a DNA. Naturally occurring nucleic acids and chemically or recombinantly produced nucleic acids may be used as a target nucleic acid.

For a method in accordance with the present teachings, at least one solid support is initially provided. In some embodiments, the solid support is a chip (e.g., a silicon chip). Silicon chips may be produced cost-effectively. In addition, additional elements (e.g., electrodes, temperature sensors, cooling and heating elements) may be integrated on the chip.

In some embodiments, at least one electrode (e.g., a gold electrode) is integrated on the surface of the support. Nucleic acids and oligonucleotides may be immobilized on gold surfaces. The electrode may be used for electrochemical reading of the label (e.g., for measuring a current).

In some embodiments, the solid support has a temperature control unit and/or a temperature sensor. Thus, a temperature of the solid support may be adjusted or measured. In some embodiments, the solid support includes cooling and/or heating elements for cooling or heating the support.

At least one capture oligonucleotide is immobilized on the solid support. The term “oligonucleotide” refers to a nucleic acid and encompasses both single-stranded and double-stranded nucleic acids. The term “oligonucleotide” encompasses both RNA molecules (e.g., oligoribonucleotides) and DNA molecules (e.g., oligodeoxyribonucleotides).

The capture oligonucleotide includes an oligonucleotide sequence that is at least sectionally complementary to the opposite-strand oligonucleotide. The section of the capture oligonucleotide where the sequence is complementary to the opposite-strand oligonucleotide may be up to about 30 nucleotides in length.

In some embodiments, the capture oligonucleotide is a single-stranded capture oligonucleotide.

In some embodiments, the capture oligonucleotide is an oligodeoxyribonucleotide.

In some embodiments, the capture oligonucleotide is immobilized on the support via a thiol group.

In some embodiments, the capture oligonucleotide is immobilized on the support via a spacer. Using the spacer allows efficient hybridization of the capture oligonucleotide to the opposite-strand oligonucleotide. The spacer may be built up from thymidine nucleotides and may be immobilized on the support via a thiol group.

In a method in accordance with the present teachings, the support is subsequently contacted under a first reaction condition with at least one opposite-strand oligonucleotide, at least one single-stranded target nucleic acid, and at least one reporter oligonucleotide having a label.

The reporter oligonucleotide includes an oligonucleotide sequence that is at least sectionally complementary to the opposite-strand oligonucleotide. The section of the reporter oligonucleotide where the sequence is complementary to the opposite-strand oligonucleotide may be up to about 30 nucleotides in length.

In some embodiments, the reporter oligonucleotide is a single-stranded reporter oligonucleotide.

In some embodiments, the reporter oligonucleotide is an oligodeoxyribonucleotide.

The reporter oligonucleotide has a label. The label may, for example, be an enzyme (e.g., an esterase) or a fluorescent dye. The label may be attached to a second end of the reporter oligonucleotide. The label may also be connected to the reporter oligonucleotide between the two ends of the reporter oligonucleotide (e.g., bonded to a base).

The opposite-strand oligonucleotide is a single-stranded oligonucleotide including at least the following oligonucleotide sequences: an oligonucleotide sequence at least sectionally complementary to the capture oligonucleotide, an oligonucleotide sequence complementary to the target nucleic acid, and an oligonucleotide sequence at least sectionally complementary to the reporter oligonucleotide. Thus, each of the capture oligonucleotide and the reporter oligonucleotide, at least sectionally, and the target nucleic acid may hybridize to the opposite-strand oligonucleotide.

The sections of the opposite-strand oligonucleotide where the sequences are complementary to the capture oligonucleotide or the reporter oligonucleotide may each be up to about 30 nucleotides in length.

The term “hybridize” refers to the attachment of a single-stranded RNA or DNA to an at least sectionally complementary single-stranded RNA or DNA with the formation of hydrogen bonds between the various complementary bases. Base pairings form between the two nucleic acid molecules in the section that is complementary. The term “base pairing” includes both Watson-Crick base pairings and non-Watson-Crick base pairings (e.g., wobble base pairing). An example of wobble base pairing is a base pairing of guanine with uracil that may form, for example, when a DNA attaches to an RNA.

In some embodiments, the oligonucleotide sequence of the opposite-strand oligonucleotide complementary to the target nucleic acid is fully complementary to the target nucleic acid. Thus, the target nucleic acid may fully hybridize to the opposite-strand oligonucleotide. The phrase “full hybridization” means that each individual base of the target nucleic acid forms a base pairing with the opposite-strand oligonucleotide. Thus, there is no individual base mismatch.

The oligonucleotide sequence of the opposite-strand oligonucleotide that is at least sectionally complementary to the capture oligonucleotide directly borders on the oligonucleotide sequence complementary to the target nucleic acid. As a result, the first end of the target nucleic acid and the free end of the capture oligonucleotide form base pairings with nucleotides of the opposite-strand oligonucleotide that are directly subsequently adjacent. The oligonucleotide sequence that is at least sectionally complementary to the reporter oligonucleotide directly borders on the other end of the oligonucleotide sequence that is complementary to the target nucleic acid. As a result, the second end of the target nucleic acid and the first end of the reporter oligonucleotide form base pairings with nucleotides of the opposite-strand oligonucleotide that are directly subsequently adjacent. As a result, the ends of the various nucleic acid molecules to be ligated may be arranged in an adjacent manner and may be directly ligated to one another without prior filling in of missing nucleotides. Since missing nucleotides may not be filled in for the ligation of the various nucleic acid molecules, the method may be carried out in a simple and rapid manner.

In some embodiments, the target nucleic acid is fully complementary to the corresponding oligonucleotide sequence of the opposite-strand oligonucleotide. The target nucleic acid may be identified and/or quantified with high specificity.

In some embodiments, the opposite-strand oligonucleotide includes an oligonucleotide sequence fully complementary to the capture oligonucleotide and/or an oligonucleotide sequence fully complementary to the reporter oligonucleotide. A stable hybridization of the opposite-strand oligonucleotide to the capture oligonucleotide and/or to the reporter oligonucleotide may result, thereby facilitating reliable identification and quantification of the target nucleic acid.

In some embodiments, the opposite-strand oligonucleotide is an oligodeoxyribonucleotide.

In some embodiments, the opposite-strand oligonucleotide, the capture oligonucleotide, and the reporter oligonucleotide are oligodeoxyribonucleotides, whereas the target nucleic acid is an RNA. During hybridization, thermodynamically stable RNA/DNA duplexes may be at least sectionally formed.

The phrase “reaction condition” refers to at least one parameter or a combination of parameters for carrying out at least one of the method acts. The parameters may include a temperature, a salt concentration, an ionic strength, a pH and/or a reagent supplement (e.g., formamide). In some embodiments, the reaction condition is selected for the melting of base pairings of capture oligonucleotide, target nucleic acid, reporter oligonucleotide, and opposite-strand oligonucleotide.

In some embodiments, the reaction condition is a temperature.

The first reaction condition is the reaction condition for hybridizing the capture oligonucleotide and the reporter oligonucleotide, at least sectionally, and the target nucleic acid to the respective sections of the opposite-strand oligonucleotide complementary thereto. The first reaction condition may be selected according to the sequence and length of the oligonucleotides and the target nucleic acid. Selection of the first reaction condition may additionally depend on whether the oligonucleotides and the target nucleic acid are RNA and/or DNA molecules.

In some embodiments, the first reaction condition includes a temperature of 42° C. in 50 mM Tris HCl (pH 7.5), 300 mM NaCl, 10 mM MgCl₂, 5 mM EDTA, and 0.025% Tween 20.

In some embodiments, the first reaction condition includes a temperature of 37° C. in 66 mM Tris HCl (pH 7.6), 50 mM NaCl, 10 mM MgCl₂, 1 mM DTT, 1 mM ATP, and 7.5% PEG6000.

The method in accordance with the present teachings also includes incubating the support under the first reaction condition.

The method in accordance with the present teachings further includes ligating the first end of the target nucleic acid to the free end of the capture oligonucleotide to covalently bond the target nucleic acid to the capture oligonucleotide, and ligating the second end of the target nucleic acid to the first end of the reporter oligonucleotide to covalently bond the target nucleic acid to the reporter oligonucleotide. The terms “ligating” and “ligation” refer to the formation of a covalent chemical bond between two single-stranded oligonucleotides on their sugar-phosphate backbone. The terms “ligating” and “ligation” include the bonding of the 3′-hydroxyl end of single-stranded RNA or DNA to the 5′-phosphoryl end of a second RNA or DNA, and also encompasses ligation of RNA and DNA. Producing the covalent bond may be catalyzed by enzymes (e.g., ligases). The enzymes may be DNA ligases may be ATP-dependent.

The method in accordance with the present teachings further includes incubating the support under a second reaction condition, such that the reporter oligonucleotide remains connected to the support only in the presence of target nucleic acid ligated at the first end and the second end of the target nucleic acid. The second reaction condition is the reaction condition for melting the base pairing of the opposite-strand oligonucleotide with the capture oligonucleotide, the target nucleic acid, and the reporter oligonucleotide. During melting, the hydrogen bonds between the various paired bases are broken. If the target nucleic acid is not ligated at both ends, the reporter oligonucleotide detaches from the support.

In some embodiments, the second reaction condition includes a higher temperature and/or a lower ionic strength than the first reaction condition. Breaking of the hydrogen bonds between the various paired bases is facilitated by the second reaction condition.

In some embodiments, the opposite-strand oligonucleotide detaches from the support by detaching from the capture oligonucleotide, the target nucleic acid, and the reporter oligonucleotide.

In some embodiments, the opposite-strand oligonucleotide remains on the support. For the opposite-strand oligonucleotide to remain on the support, the second reaction condition has a lower temperature and/or a lower ionic strength as compared to the reaction condition for detachment of the opposite-strand oligonucleotide from the support. Due to an energy gain resulting from the two ligations, the presence of the opposite-strand oligonucleotide connected by base pairings to the capture oligonucleotide, the target nucleic acid, and the reporter oligonucleotide has an energetically stabilizing effect. The stabilization makes facilitates reading of the label on the support under milder conditions as compared to in the absence of the opposite-strand oligonucleotide.

In some embodiments, the second reaction condition includes a temperature of 52° C. in 50 mM Tris HCl (pH 7.5) and 150 mM NaCl.

In some embodiments, the second reaction condition includes a temperature of 50° C. in 25 mM Tris HCl (pH 7.5) and 25 mM NaCl.

The method in accordance with the present teachings further includes reading the label of the reporter oligonucleotide on the support. Using the presence of the label on the support, it may be determined that the target nucleic acid has been ligated at its two ends. The first end of the target nucleic acid has been ligated to the free end of the capture oligonucleotide and the second end of the target nucleic acid has been ligated to the first end of the reporter oligonucleotide. Since the reporter oligonucleotide remains connected to the support only in the presence of target nucleic acid ligated at its two ends, the target nucleic acid is identified by detecting the label of the reporter oligonucleotide on the support. Detection of the label on the support may also be used to quantify the target nucleic acid.

The sequence of the acts in a method in accordance with the present teachings is not restricted. In some embodiments, the individual acts may be carried out in different sequences than the sequences described herein. In other embodiments, the individual acts may be carried out in the same sequence as described herein.

In some embodiments, two or more acts of a method in accordance with the present teachings may be combined together. As a result, the method may be carried out more efficiently. For example, in some embodiments, act (b) and act (d) are combined together. To effect this combination, the reagents used for the ligation (e.g., ligase) may be pre-added to the support in act (b). In some embodiments, act (b) and act (c) are carried out in a single act.

In accordance with the present teachings, the target nucleic acid may be directly identified and/or quantified. In some embodiments, the target nucleic acid may not be chemically or enzymatically modified. Since certain target nucleic acids may be modified, the quantification of modified target nucleic acids may lead to inaccurate results. These inaccuracies may be avoided by the direct detection of the target nucleic acid. Furthermore, artifacts that may occur during modification of the target nucleic acid are avoided.

A method in accordance with the present teachings has high sensitivity. As a result, even low amounts of the target nucleic acid may be detected. The target nucleic acid may not be amplified before identification, such that incorrect nucleotides are not integrated into the target nucleic acid, thereby increasing the specificity of the method.

Since both ends of the target nucleic acid are involved in its identification, a method in accordance with the present teachings has high selectivity.

Owing to the two ligations, the reporter oligonucleotide is covalently bonded to the support via the target nucleic acid and the capture oligonucleotide. The detection of the label of the reporter oligonucleotide is independent of the sequence and length of the oligonucleotides and the target nucleic acid. There is a temperature dependence of attachment of reporter oligonucleotide, target nucleic acid, and capture oligonucleotide to the opposite-strand oligonucleotide. As a result, the temperature when the label is read may be optimally adjusted to the nature of the label used, thereby contributing to the high sensitivity of the method. Furthermore, the stable covalent bonding of the reporter oligonucleotide to the support allows robust and quantitatively analyzable reading of the label.

In some embodiments, washing of the support is additionally carried out before act (d) and/or during act (e) and/or before act (f). Washing removes detached opposite-strand oligonucleotide from the support. In addition, nonbonded reporter oligonucleotides and nonbonded nucleic acids are removed from the support, thereby preventing reattachment of the opposite-strand oligonucleotide to the capture oligonucleotide and reattachment of the reporter oligonucleotide to the opposite-strand oligonucleotide during reading of the label. As a result, there is increased specificity of detection of the label on the support and, therefore, increased specificity of identification of the target nucleic acid.

In some embodiments, washing of the support is carried out before act (d) under a stringent reaction condition.

The phrase “stringent reaction condition” refers to a reaction condition wherein only fully hybridized target nucleic acid remains connected to the opposite-strand oligonucleotide. By contrast, target nucleic acid molecules with a sequence having one or more base mismatches with respect to the corresponding oligonucleotide sequence of the opposite-strand oligonucleotide detach from the opposite-strand oligonucleotide. By washing the support under a stringent reaction condition before ligation, high specificity of the method is achieved.

In some embodiments, the target nucleic acid is an RNA and, in some embodiments, a microRNA (miRNA). A miRNA is a noncoding, single-stranded RNA of about 17 to about 25 nucleotides in length that is effective in the posttranscriptional regulation of gene expression. A miRNA attaches highly specifically to complementary sequences of mRNA molecules and, as a result, regulates the translation of the mRNA into the corresponding protein.

In some embodiments, the target nucleic acid is a single-stranded small interfering RNA (siRNA). Similarly to the miRNAs, siRNAs may also be involved in the posttranscriptional regulation of gene expression by attaching specifically to complementary sequences of mRNA molecules and regulating the translation of the mRNA into the corresponding protein.

In some embodiments, a T4 DNA ligase is used for the ligation. A T4 DNA ligase is an enzyme that catalyzes the ATP-dependent ligation of a 3′-hydroxyl end of a first nucleic acid molecule to a directly subsequently adjacent 5′-phosphoryl end of a second nucleic acid molecule in double-stranded DNA, RNA/DNA or RNA molecules. The catalysis produces a covalent phosphodiester bond.

In some embodiments, phosphorylation of one end of the target nucleic acid, the capture oligonucleotide, and/or the reporter oligonucleotide is carried out. The term “phosphorylation” refers to the attachment of a phosphate moiety to the 5′-hydroxyl end of RNA or DNA. The ligation of two directly subsequently adjacent oligonucleotides or nucleic acid molecules uses a 5′-phosphoryl end on the second nucleic acid molecule. The presence of a 5′-phosphoryl end of the second nucleic acid molecule depends inter alia on its synthesis. A missing 5′-phosphoryl end may be attached as a result of the phosphorylation so that the capture oligonucleotide may be ligated to the target nucleic acid and the target nucleic acid to the reporter oligonucleotide.

In some embodiments, the phosphorylation is an enzymatic phosphorylation.

In some embodiments, the phosphorylation is carried out by contacting the molecule to be phosphorylated with a T4 polynucleotide kinase. A T4 polynucleotide kinase is an enzyme that catalyzes the phosphorylation of 5′-hydroxyl ends of RNA or DNA molecules.

In some embodiments, the label of the reporter oligonucleotide is an enzyme.

In some embodiments, the enzyme is an esterase (e.g., in some embodiments, a thermostable esterase). Due to thermostability, the enzymatic activity of the esterase is preserved during the method even at increased temperatures. Thus, an esterase-labeled reporter oligonucleotide may be contacted with the support as early as at the start of the method By contrast, reading of the label is only carried out in the last act of the method.

In some embodiments, the esterase is the thermostable esterase 2 from Alicyclobacillus acidocaldarius. The esterase 2 is built up from a single protein chain and may therefore be easily coupled to the reporter oligonucleotide.

In some embodiments, the enzyme is covalently bonded to the reporter oligonucleotide. As a result, the enzyme is stably connected to the reporter oligonucleotide and cannot detach from the reporter oligonucleotide owing to washing processes or the changing temperatures. Reliable identification and quantification of the target nucleic acid may therefore be provided.

In some embodiments, the enzyme is bonded to an amino group of the reporter oligonucleotide via a thiol group. Esterase 2 from Alicyclobacillus acidocaldarius may be bonded in a directed manner to the reporter oligonucleotide via a cysteine. The directed bonding provides accessibility of the active site of esterase 2 to substrate. The directed covalent bonding of esterase 2 to an oligodeoxynucleotide may be achieved as described, for example, by Wang et al. in Biosensors and Bioelectronics, 2007, 22, 1798-1806.

In some embodiments, the label is a fluorescent dye that may be covalently bonded to the reporter oligonucleotide. Use of the fluorescent dye facilitates simple and rapid reading of the label. Furthermore, no substrate is involved in the reading process, and the method may be carried out in a cost-effective manner.

In some embodiments, the label is read electrochemically. During an electrochemical reading, substrates are converted to redox-active reaction products on the label (e.g., in some embodiments, an enzyme). As a result, a current or a change in voltage may be measured at one electrode.

In some embodiments, reading is achieved by redox cycling of p-aminophenol and quinonimine. For this purpose, the label may be an enzyme that hydrolyzes the substrate p-aminophenylbutyrate to the redox-active reaction product p-aminophenol. By reversing the polarity of the electrode potentials, redox cycling of p-aminophenol and quinonimine is achieved and electrons are continually produced. As a result, a current may be measured at the electrodes. The current indicates the detection of the enzyme label and is also a measure of the conversion of substrate to reaction product on the enzyme label. Electrochemically reading the label by measuring the current may therefore be used for identifying and/or quantifying the target nucleic acid.

In some embodiments, reading of the label on the support is carried out at a reading temperature. The reading temperature is within a range of high enzymatic activity of the enzyme. If esterase 2 from Alicyclobacillus acidocaldarius is used as label, the reading temperature may be about 30° C.

In some embodiments, reading of the label on the support is carried out optically. Optically reading the label is simple and cost-effective. If the label is an enzyme, the substrate of the enzyme and the reaction product obtained by the reaction may have different optical properties. For example, the reaction product exhibits different light absorption with respect to the substrate. The reaction product may then, for example, be detected using a spectrophotometer. If the label is a fluorescent dye, the label may be read using a fluorescence photometer.

In some embodiments, reading of the label is carried out in a position-specific manner. As a result, multiple different target nucleic acids may be identified and/or quantified in parallel in a single method. As a result, multiple samples to be investigated may also be analyzed using a single method.

In some embodiments, a first reading of the label on the support is carried out before act (d) and/or act (e), and/or a second reading of the label on the support is carried out during act (f). For the first reading of the label, a reference value is obtained, whereas for the second reading of the label, a measured value is obtained. The measured value may be normalized to the reference value. As a result, data obtained from various supports may be compared to one another. The reference value may, for example, be a measure of the number of capture oligonucleotides immobilized on the support. If fewer capture oligonucleotides are immobilized on a first support than on a second support, then both the reference value and the measured value of the first support are lower. Therefore, calibration of the individual supports may be carried out.

A quotient formed from the measured value and the reference value remains approximately constant for differing numbers of immobilized capture oligonucleotides on different supports. As a result, scatterings of measured values obtained from various supports may be eliminated. Therefore, better reproducibility and lower standard deviations for the values obtained during reading of the label may also be achieved.

Furthermore, calibration of individual positions on the same support is facilitated.

In some embodiments, the first reading and the second reading are carried out under an identical reaction condition. As a result, the enzyme activity that is dependent on the reaction condition (e.g., temperature) is identical during the first reading and the second reading.

In accordance with the present teachings, a kit is provided for the detection of at least one single-stranded target nucleic acid in a sample. The kit includes at least one solid support that includes at least one capture oligonucleotide immobilized thereon, at least one reporter oligonucleotide that includes a label, and at least one opposite-strand oligonucleotide. The opposite-strand oligonucleotide includes an oligonucleotide sequence that is at least sectionally complementary to the capture oligonucleotide, an oligonucleotide sequence complementary to the target nucleic acid in the sample, and an oligonucleotide sequence at least sectionally complementary to the reporter oligonucleotide. The opposite-strand oligonucleotide is configured for hybridizing at least sectionally each of the capture oligonucleotide and the reporter oligonucleotide, and is further configured for hybridizing the target nucleic acid to the opposite-strand oligonucleotide. A first end of the target nucleic acid and a free end of the capture oligonucleotide are configured to form base pairings with adjacent nucleotides of the opposite-strand oligonucleotide. A second end of the target nucleic acid and a first end of the reporter oligonucleotide are configured to form base pairings with adjacent nucleotides of the opposite-strand oligonucleotide. The label of the reporter oligonucleotide indicates the presence of the target nucleic acid in the sample.

The sample may be a mixture of the target nucleic acid with other nucleic acids and/or proteins. The sample may additionally be a lysate obtained from cells. The cells may be human, animal, plant or bacterial cells. In some embodiments, the cells originate from a biopsy taken from a patient. If the patient has a condition or a suspected condition that occurs at an elevated level or a decreased level of the target nucleic acid, the kit may be used for the diagnostic detection of the target nucleic acid in the biopsy. In some embodiments, the condition is a cancer (e.g., breast cancer, lung cancer, or leukemia). In some embodiments, the condition is caused by a disorder of the immune system.

In some embodiments, the kit additionally includes at least one nucleic acid ligase (e.g., a T4 DNA ligase) for ligating the first end of the target nucleic acid to the free end of the capture oligonucleotide to covalently bond the target nucleic acid to the capture oligonucleotide, and for ligating the second end of the target nucleic acid to the first end of the reporter oligonucleotide to covalently bond the target nucleic acid to the reporter oligonucleotide.

In some embodiments, the label is an enzyme and the kit additionally includes at least one substrate specific for the enzyme.

In some embodiments, the target nucleic acid is an RNA. In some embodiments, the target nucleic acid is an miRNA.

Exemplary embodiments of a method in accordance with the present teachings will now be illustrated with reference to schematic drawings.

### BRIEF DESCRIPTION OF THE DRAWINGS

FIG. 1 shows a schematic illustration of the identification of a single-stranded target nucleic acid (9) using a method in accordance with the present teachings.

FIG. 2 shows a schematic illustration of a method in accordance with the present teachings involving a nucleic acid (23) where the sequence does not match the sequence of the miR-16 to be identified.

FIG. 3 shows a schematic illustration of a method in accordance with the present teachings using a nucleic acid (25) where the sequence only sectionally matches the sequence of the miR-16 to be identified.

FIG. 4 shows a schematic illustration of a method in accordance with the present teachings including calibration of the support (1).

### DETAILED DESCRIPTION

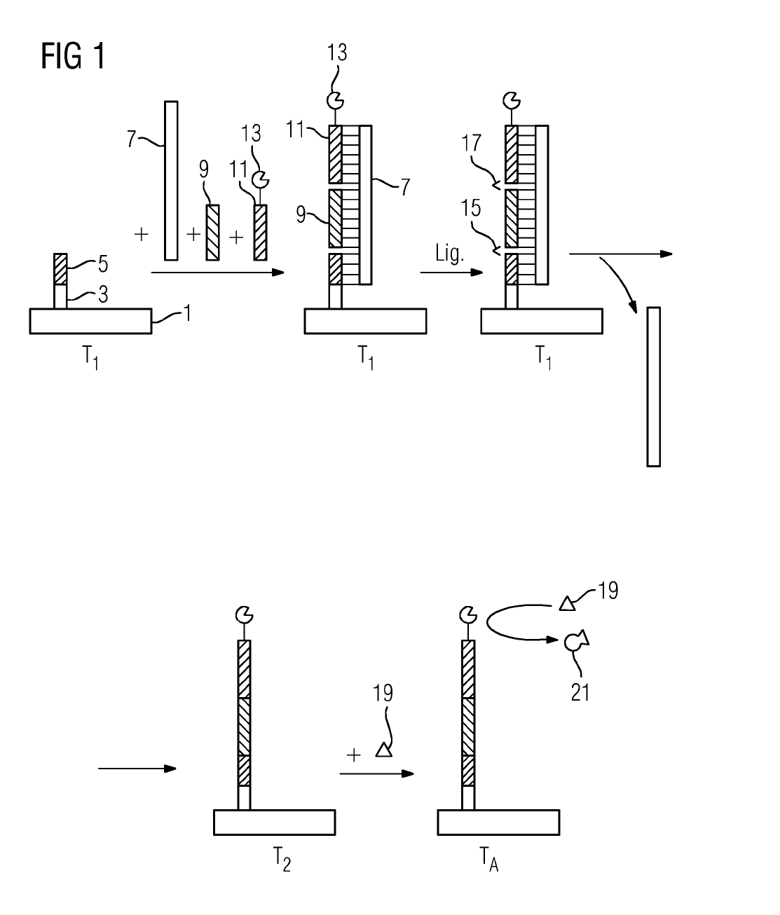

FIG. 1 shows an exemplary identification of a single-stranded target nucleic acid (9) using a method in accordance with the present teachings. The target nucleic acid (9) is miR-16. The miR-16 is an miRNA that may reduce the expression of the antiapoptotic protein Bcl-2 in lymphocytes. A silicon chip having two integrated gold electrodes is used as a solid support (1). A single-stranded capture oligonucleotide (5) is immobilized on the solid support (1) by a spacer (3). An opposite-strand oligonucleotide (7), the miR-16, and a single-stranded reporter oligonucleotide (11) are contacted with the support (1) at a first temperature of 42° C. The reporter oligonucleotide (11) has a thermostable esterase 2 from Alicyclobacillus acidocaldarius as a label (13). The label (13) is covalently bonded to the reporter oligonucleotide (11). The opposite-strand oligonucleotide (7) includes three oligonucleotide sequences arranged such that the sequences directly border on one another.

One oligonucleotide sequence is fully complementary to the capture oligonucleotide (5). Next to this sequence is an oligonucleotide sequence that is fully complementary to the miRNA. This sequence is followed by an oligonucleotide sequence that is fully complementary to the reporter oligonucleotide (11). The support (1) is incubated at 42° C. for 20 min. The capture oligonucleotide (5), the miR-16, and the reporter oligonucleotide (11) fully hybridize to the respective sections of the opposite-strand oligonucleotide (7) complementary thereto. As shown in FIG. 1, the result of the arrangement of the oligonucleotide sequences on the opposite-strand oligonucleotide (7) is that a lower end of the miR-16 and a free upper end of the capture oligonucleotide (5), and also an upper end of the miR-16 and a lower end of the reporter oligonucleotide (11), each hybridize to adjacent nucleotides of the opposite-strand oligonucleotide (7). A T4 DNA ligase is contacted with the support (1). The ligase ligates together, in a first ligation (15), the adjacent ends of the capture oligonucleotide (5) and the miR-16. In a second ligation (17), the adjacent ends of the miR-16 and the reporter oligonucleotide (11) are ligated together. As a result, the miR-16 is, at the lower end, covalently bonded to the capture oligonucleotide (5) and, at the upper end, covalently bonded to the reporter oligonucleotide (11). The support (1) is incubated at a second temperature of 52° C. for 10 minutes. This incubation melts the base pairing of the opposite-strand oligonucleotide (7) with the capture oligonucleotide (5), the miRNA, and the reporter oligonucleotide (11). As a result, the opposite-strand oligonucleotide (7) detaches. The support (1) is washed at 52° C. with a salt-containing buffer solution to remove the detached opposite-strand oligonucleotide (7). The support (1) is brought to a reading temperature of 30° C. and the esterase 2 on the support (1) is analyzed. This reading is carried out electrochemically. Thus, the support (1) is contacted with a substrate (19) of the esterase 2, p-aminophenylbutyrate. The esterase 2 converts the p-aminophenylbutyrate to a redox-active reaction product (21), p-aminophenol. Due to redox cycling of p-aminophenol and quinonimine, current is generated that is measured at the gold electrode. Measurement of the current serves as detection of the esterase 2 on the support (1) and indicates the presence of the miR-16.

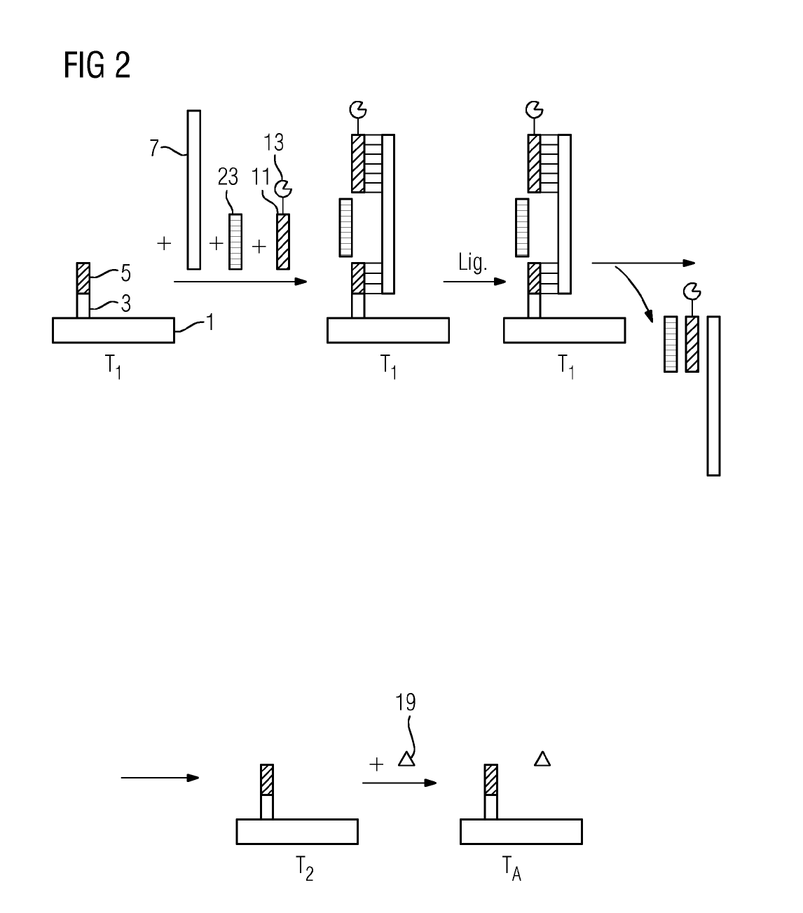

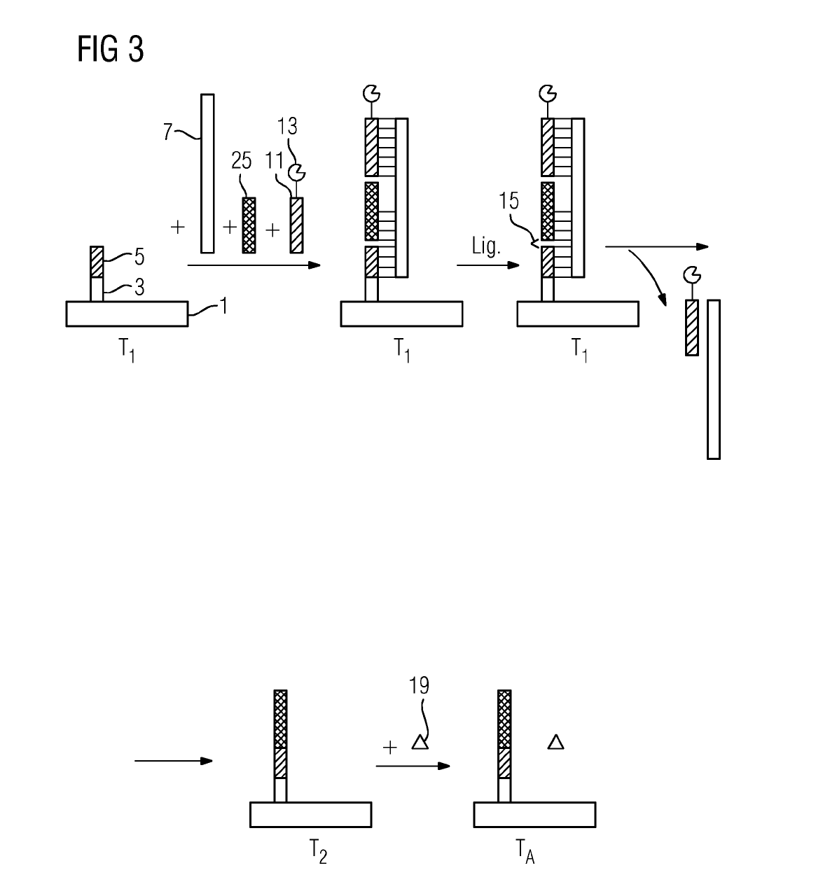

FIG. 2 shows a method in accordance with the present teachings using a nucleic acid (23) where the sequence does not match the sequence of the miR-16 to be identified. FIG. 3 shows a method in accordance with the present teachings using a nucleic acid (25) where the sequence only sectionally matches the sequence of the miR-16 to be identified. The nucleic acid (23 or 25) is used as a negative control for verifying a method in accordance with the present teachings. FIG. 2 shows that a nucleic acid (23) wherein the sequence does not match the sequence of the miR-16 does not hybridize to the opposite-strand oligonucleotide (7) during incubation of the support (1) at 42° C. Therefore, the T4 DNA ligase cannot ligate the ends of the capture oligonucleotide (5) and the reporter oligonucleotide (11) to the ends of the nucleic acid (23). During incubation and washing of the support (1) at 52° C., the opposite-strand oligonucleotide (7) detaches from the capture oligonucleotide (5) and the reporter oligonucleotide (11). Because of the lack of covalent bonding of the reporter oligonucleotide (11) to the support (1) via the target nucleic acid (9) and the capture oligonucleotide (5), the reporter oligonucleotide (11) is removed from the support (1), such that no esterase 2 remains on the support (1). As a result, no current is measured when reading the esterase 2.

FIG. 3 shows that a nucleic acid (25) where the sequence sectionally matches the sequence of the miR-16 hybridizes to the opposite-strand oligonucleotide (7) in the section matching the sequence of the miRNA, but not fully, during incubation of the support (1) at 42° C. The first end of the nucleic acid (25) and the free end of the capture oligonucleotide (5) hybridize to adjacent nucleotides of the opposite-strand oligonucleotide (7). The T4 DNA ligase ligates together the adjacent ends of the capture oligonucleotide (5) and the nucleic acid (25).

The nucleic acid (25) is covalently bonded to the capture oligonucleotide (5). Due to a lack of complementarity, the second end of the nucleic acid (25) does not hybridize to the opposite-strand oligonucleotide (7), such that the T4 DNA ligase does not ligate the second end of the nucleic acid (25) to the first end of the reporter oligonucleotide (11). During incubation and washing of the support (1) at 52° C., the opposite-strand oligonucleotide (7) detaches from the capture oligonucleotide (5), the nucleic acid (25), and the reporter oligonucleotide (11). Due to the lack of covalent bonding of the reporter oligonucleotide (11) to the nucleic acid (25), the reporter oligonucleotide (11) is also removed from the support (1), such that no esterase 2 remains on the support (1). As a result, no current is measured when reading the esterase 2.

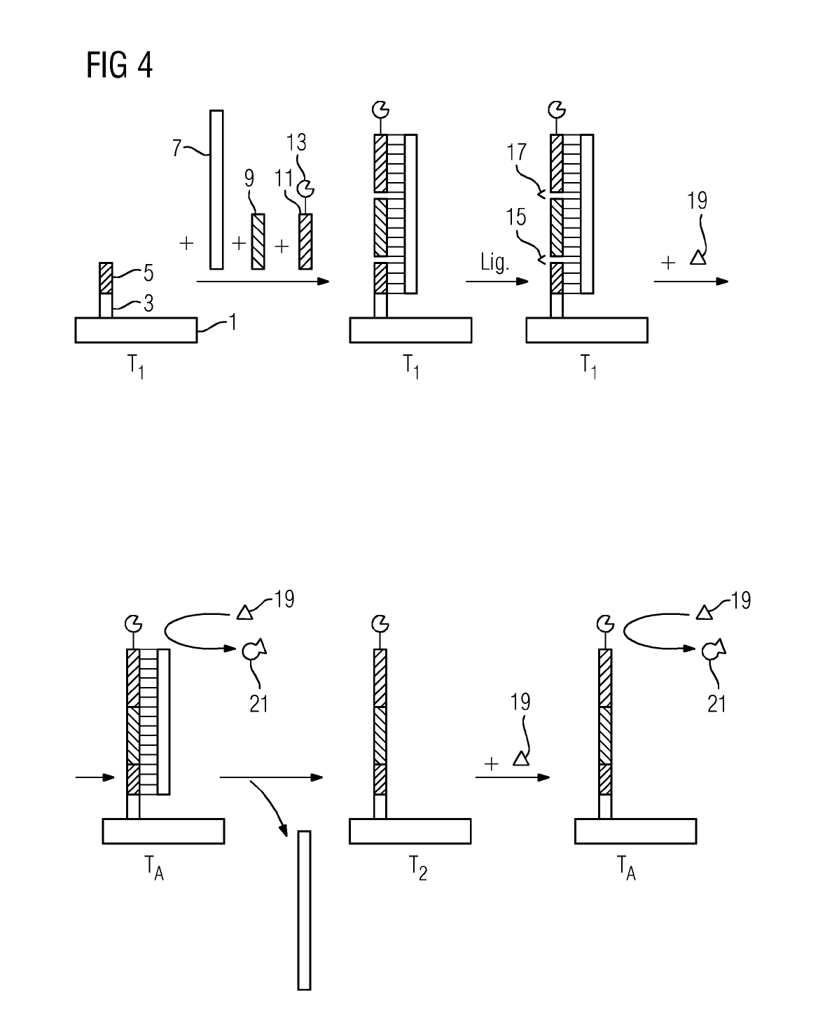

FIG. 4 shows an embodiment of a method in accordance with the present teachings that includes calibration of the support (1). The reading temperature of 30° C. is set after ligation of the respective adjacent ends of the capture oligonucleotide (5), the miR-16, and the reporter oligonucleotide (11), and the esterase 2 on the support (1) is analyzed for the first time. In this analysis, a reference value is obtained. The support (1) is incubated at 52° C. At this temperature, the opposite-strand oligonucleotide (7) separates from the capture oligonucleotide (5), the miR-16, and the reporter oligonucleotide (11). A temperature of 30° C. is set again and the esterase 2 on the support (1) is analyzed for the second time. In this analysis, a measured value is obtained. The measured value is normalized to the reference value, such that the data obtained from various supports may be compared to one another.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding claim—whether independent or dependent—and that such new combinations are to be understood as forming a part of the present specification.

1. A method for identifying and quantifying at least one single-stranded target nucleic acid, the method comprising:
(a) providing at least one solid support comprising at least one capture oligonucleotide immobilized thereon;
(b) contacting the support under a first reaction condition with at least one opposite-strand oligonucleotide, at least one single-stranded target nucleic acid, and at least one reporter oligonucleotide that comprises a label;
wherein the opposite-strand oligonucleotide comprises an oligonucleotide sequence at least sectionally complementary to the capture oligonucleotide, an oligonucleotide sequence complementary to the target nucleic acid, and an oligonucleotide sequence at least sectionally complementary to the reporter oligonucleotide;
wherein the opposite-strand oligonucleotide is configured for hybridizing at least sectionally each of the capture oligonucleotide and the reporter oligonucleotide, and is further configured for hybridizing the target nucleic acid to the opposite-strand oligonucleotide;
wherein a first end of the target nucleic acid and a free end of the capture oligonucleotide are configured to form base pairings with adjacent nucleotides of the opposite-strand oligonucleotide; and
wherein a second end of the target nucleic acid and a first end of the reporter oligonucleotide are configured to form base pairings with adjacent nucleotides of the opposite-strand oligonucleotide;
(c) incubating the support under the first reaction condition;
(d) ligating the first end of the target nucleic acid to the free end of the capture oligonucleotide to covalently bond the target nucleic acid to the capture oligonucleotide, and ligating the second end of the target nucleic acid to the first end of the reporter oligonucleotide to covalently bond the target nucleic acid to the reporter oligonucleotide;
(e) incubating the support under a second reaction condition, such that the reporter oligonucleotide remains connected to the support when the target nucleic acid is ligated at the first end and the second end thereof; and
(f) reading the label of the reporter oligonucleotide on the support.

2. The method of claim 1, further comprising washing the support at a stage of the method selected from the group consisting of before (d), during (e), before (f), and combinations thereof.

3. The method of claim 2, wherein the washing of the support occurs before (d) under a stringent reaction condition.

4. The method of claim 1, wherein the target nucleic acid comprises an RNA.

5. The method of claim 1 further comprising phosphorylating a material selected from the group consisting of the target nucleic acid, the capture oligonucleotide, the reporter oligonucleotide, and combinations thereof.

6. The method of claim 1 wherein the label comprises an enzyme.

7. The method of claim 1 further comprising reading the label electrochemically.

8. The method of claim 1 further comprising:
performing a first reading of the label on the support at a time selected from the group consisting of before (d), before (e), and a combination thereof;
performing a second reading of the label on the support during (f); or
performing a first reading of the label on the support at a time selected from the group consisting of before (d), before (e), and a combination thereof, and performing a second reading of the label on the support during (f).

9. The method of claim 8, wherein the first reading and the second reading are carried out under an identical reaction condition.

10. A kit for detecting at least one single-stranded target nucleic acid in a sample, the kit comprising
at least one solid support comprising at least one capture oligonucleotide immobilized thereon;
at least one reporter oligonucleotide comprising a label; and
at least one opposite-strand oligonucleotide, the opposite-strand oligonucleotide comprising an oligonucleotide sequence at least sectionally complementary to the capture oligonucleotide, an oligonucleotide sequence complementary to the target nucleic acid in the sample, and an oligonucleotide sequence at least sectionally complementary to the reporter oligonucleotide;
wherein the opposite-strand oligonucleotide is configured for hybridizing at least sectionally each of the capture oligonucleotide and the reporter oligonucleotide, and is further configured for hybridizing the target nucleic acid to the opposite-strand oligonucleotide;
wherein a first end of the target nucleic acid and a free end of the capture oligonucleotide are configured to form base pairings with adjacent nucleotides of the opposite-strand oligonucleotide;
wherein a second end of the target nucleic acid and a first end of the reporter oligonucleotide are configured to form base pairings with adjacent nucleotides of the opposite-strand oligonucleotide; and
wherein the label of the reporter oligonucleotide is configured to indicate a presence of the target nucleic acid in the sample.

11. The method of claim 2, wherein the target nucleic acid comprises an RNA.

12. The method of claim 3, wherein the target nucleic acid comprises an RNA.

13. The method of claim 1, wherein the target nucleic acid comprises an miRNA.

14. The method of claim 2 further comprising phosphorylating a material selected from the group consisting of the target nucleic acid, the capture oligonucleotide, the reporter oligonucleotide, and combinations thereof.

15. The method of claim 3 further comprising phosphorylating a material selected from the group consisting of the target nucleic acid, the capture oligonucleotide, the reporter oligonucleotide, and combinations thereof.

16. The method of claim 4 further comprising phosphorylating a material selected from the group consisting of the target nucleic acid, the capture oligonucleotide, the reporter oligonucleotide, and combinations thereof.

17. The method of claim 1 wherein the label comprises an esterase.

18. The method of claim 1 wherein the label comprises a thermostable esterase.

19. The method of claim 18 wherein the thermostable esterase is covalently bonded to the reporter oligonucleotide.

20. The method of claim 7 wherein the reading of the label comprises redox cycling of p-aminophenol and quinonimine.
